(19) United States
(12) Patent Application Publication
(10) Pub. No.: US 2005/0148931 A1
(43) Pub. Date: Jul. 7, 2005

(54) Safety syringe

(76) Inventor: Paul R. Juhasz, Setauket, NY (US)

Correspondence Address: Paul R. Juhasz, P.O. Box 309, Farmingville, NY, 11738, US

(21) Appl. No.: 10/972,708

(22) Filed: Oct. 25, 2004

Related U.S. Application Data
(60) Provisional application No. 60/515,439, filed on Oct. 30, 2003.

(51) Int. Cl. A61M 5/00

(52) U.S. Cl. 604/110

(57) ABSTRACT

Preferred embodiments of the present invention provide a needle assembly for use with a syringe having a needle that is protected by the needle assembly after use. In one embodiment, the needle is retracted into the needle assembly. In an alternative embodiment, the needle assembly is biased to protect the needle after use. Among other disclosed embodiments is a retractable needle.

### BACKGROUND

This invention relates generally to syringe devices and more particularly to a syringe that is adapted to shielding a needle attached thereto so as to prevent accidental needle strike when the syringe is not being used.

Health care and other individuals who use or come into contact with hypodermic needle type syringes are susceptible to accidental and potentially infectious needle strike due to careless handling and disposing of the syringes after being used. The use or contact can occur in a medical setting, such as a hospital, such as when administering a medication or drawing blood; at home, such as in connection with an allergy or insulin shot; or in any other setting where use or disposal of a syringe may take place. The use or contact can involve a pre-filled, non-prefilled, or other syringe device.

In U.S. Pat. No. 5,061,251, this inventor patented a spring-loaded shielded syringe in which a spring so biases a shield as to protect the needle when the device is not being used. Over time, generally three industrial approaches were taken to solving the problem of needle strike.

The first approach was a spring-less shield that could be manually retracted to expose the needle for use and then manually returned to the shielding position to protect against needle strike after it has been used. While easy to implement, it requires the user to return the shield to the protection position after use. Failure to do so, such as in the heat of a surgery or accidental oversight, can lead to instances of needle strike.

The second approach employed a spring-loaded shield as taught by this inventor in the '251 patent. This approach has been has been followed in U.S. Pat. No. 6,613,022 and U.S. Pat. No. 6,616,639.

In the third approach, a syringe was so modified as to allow the needle to be retracted into the barrel after use. A number of companies have adopted this approach as a solution to protecting against needle strike.

While there is a bias in the industry toward providing safer needles, there are also technical and financial limitations imposed on what the industry can do to make needles safe. Any solution that requires modifications to be made to the design of the syringe can require significant capital investment. In addition to refining and testing the design modifications, studies and filings are needed in order to secure regulatory approvals to use these modified devices with humans. A considerable amount of retooling and other manufacturing costs are also incurred before the redesigned syringe can be commercialized. These significant costs act to retard the migration of the industry toward a safer syringe.

In the late 1990's, California led the way in requiring manufacturers to make syringes more effective against needle strike. Other states have followed since. Such legislation is believed to have created considerable bias on the industry to design and commercializee safer syringes. Since then, a greater number of safer syringes have been made available to the public.

Yet cost continues to drive the syringe commercialization process. The costs of redesigning, retooling, and recertifying syringes employing more sophisticated safety technology solutions seems to continue to impose barriers to a wider roll-out of syringes that clearly appear to be safer.

Another factor influencing the syringe design process are industrial standards that have been adopted by the health care industry so as to allow products of different companies to be interchangeably used. One such standard is the Luer Lok thread on the collar of a syringe barrel that allows needles and syringes from a variety of manufacturers to be interchangeably used. Many of the more elegant technical solutions to needle strike fail to accommodate these industrial standards, instead focusing on finding the most sophisticated technical solution to the problem. The results are elegant technical solutions that require such costly modifications as to make them commercially less attractive as solutions to the needle strike problem.

One of the more recent improvements on the solution employing a retractible needle is described in European Patent Application EP1092443A2 and PCT WO00/37131. In these filings, a needle assembly and syringe have been modified to provide a safer syringe. In particular, the needle actually retracts from the needle assembly into the barrel of the syringe and more particularly into a cavity formed in the plunger that penetrates the bore of the syringe. Elegant a technical solution as it appears to be, this design requires significant modifications to be made to both the syringe and the needle assembly. Such modifications would appear to be inconsistent with current standards employed in the industry such as the conventional Luer Lok that allows needle assemblies of companies to be interchanged with syringes provided by other companies. The capital intensiveness required to make these modifications would suggest that this more recent improvement will not be widely employed. See also U.S. Pat. No. 6,273,870 where the syringe and its barrel have been so modified as to allow a needle to be retracted into the barrel of the syringe.

There is a need for a greater availability of syringes to protect health care workers and other individuals who use or come into contact with hypodermic needle type syringes. There is a need for a greater availability of syringes that protect against accidental and potentially infectious needle strike due to careless handling and disposing of syringes, whether the use or contact occurs in a medical setting, such as a hospital; at home, such as in connection with an allergy or insulin shot; or in any other setting where use or disposal of a syringe may take place.

There is a need for a safety syringe design that is technically simple and less cost prohibitive. There is a need for a simple safety syringe design that can be widely adopted by the industry. There is a need for a safety syringe that is compatible with features of syringes that have become industrial standards. There is a need for a safety syringe design that lends itself to retrofitting to existing syringe designs in use by the industry. The present invention is believed to address these and other problems.

### SUMMARY

Preferred embodiments of the present invention provide a needle assembly for use with a syringe having a needle that is protected by the needle assembly after use. In one embodiment, the needle is retracted into the needle assembly. In an alternative embodiment, the needle assembly is biased to protect the needle after use.

In one embodiment, the needle assembly comprises: a housing having a first part and a second part, said first part defining a cavity therein and said second part being movable with respect to said first part; a needle attached to said second part; a spring located between said first part and said second part; wherein on application of a force, said second part moves with respect to said first part allowing said spring to retract the needle into said cavity defined by said first part of said housing; and wherein, after retraction, said needle comes to a rest inside said cavity, said housing shielding said needle against needle strike.

In an alternative embodiment, the needle assembly comprises: a housing having a first part and a second part, said first part defining a conduit therein and being movable with respect to said second part; a needle attached to said second part; a spring located between said first part and said second part; wherein on application of a force, said first part moves with respect to said second part allowing said spring to so bias the first part as to shield the needle by said conduit defined by said first part of said housing; and wherein, after so biasing the first part, said needle sits inside said conduit, said housing shielding said needle against needle strike.

As an illustrative example of one embodiment, the needle assembly comprises a first hub having a first end, a second end, and a conduit connecting the two, said first end defining a cavity, said hub having an inner portion and an outer portion, said inner portion being movable with respect to the outer portion on the application of a force; a second hub having a first end, a second end, and a conduit connecting the two for receiving said first hub; a spring disposed between said first hub and said second hub; a needle having a first end, a second end, and a lumen running therethrough, said first end of said needle being so connected to said second end of the first hub as to allow there to be communication between the lumen and said conduit of said first hub; wherein on application of a force, the inner portion of the first hub is allowed to move with respect to the outer portion of the first hub, causing said spring to retract the needle into said cavity defined by said first hub of said assembly; and wherein said needle comes to rest inside said cavity of said first hub.

Preferably, the inner and outer portions are an integrally molded structure that is weakened along the boundary between the two by score lines, recesses, or material discontinuities. Alternatively, inner and outer portions may be formed from two separate materials and held together by a breakable adhesive, friction or snap fit, for example. Movement of the inner portion with respect to the outer portion may be on the application of a force, such as, for example, by striking the head of a needle against a surface, turning the second hub with respect to the first hub (or vice-versa), or activation of a release mechanism.

The assembly is made with few parts. When adapted to a Luer Lok fitting, the assembly may be attachable to conventional syringes provided with a Luer Lok connector.

When the force applied to move the inner and outer portions of the first hub is the striking of the needle against a surface, a strike pad may also be used to provide the strike surface for activating the retraction of the needle into the assembly.

The above illustrate some embodiments with numerous other embodiments further disclosed.

### DESCRIPTION OF THE DRAWINGS

FIG. 1 shows an aerial rendition of the syringe device of this invention.

FIG. 2 shows a cross-section of a preferred embodiment of a first hub of the safety syringe of FIG. 1.

FIG. 3 shows a cross-section of a preferred embodiment of a second hub of the safety syringe of FIG. 1.

FIG. 4 shows a cross-section of the safety syringe of FIG. 1 with a needle assembly of this invention assembled using first and second hubs of FIGS. 2 and 3 with the safety syringe shown prior to retraction of the needle into the needle assembly.

FIG. 5 shows a striking of the needle against a strike pad in order to effect a retraction of the needle into the needle assembly of FIG. 4.

FIG. 6 shows a cross-section of the needle assembly of FIG. 4 after the needle has come to rest inside the needle assembly after the striking action of FIG. 5.

FIG. 7 shows a further embodiment of the needle assembly of FIG. 4.

FIG. 8 shows a cross-section of a first hub of a third embodiment of the safety syringe of FIG. 1.

FIG. 9 shows a cross-section of a second hub of a third embodiment of the safety syringe of FIG. 1.

FIG. 10 shows a cross-section of the safety syringe of FIG. 1 with a needle assembly of this invention assembled using first and second hubs of FIGS. 8 and 9 with the needle assembly shown prior to retraction of the needle into the needle assembly.

FIG. 11 shows a cross-section of the safety syringe of FIG. 10 after the needle has come to rest inside the needle assembly after the striking action of FIG. 5.

FIG. 12 shows a perspective view of an inventive needle of the needle assembly of this invention.

FIG. 13 shows a cross-section of a first hub of a fourth embodiment of the safety syringe of FIG. 1.

FIG. 14 shows a cross-section of a second hub of a fourth embodiment of the safety syringe of FIG. 1.

FIG. 15 shows a cross-section of the safety syringe of FIG. 1 with a needle assembly according to this invention assembled using first and second hubs of FIGS. 13 and 14 with the needle assembly shown prior to retraction of the needle into the needle assembly.

FIG. 16 shows a cross-section of the needle assembly of FIG. 15 after the needle has come to rest inside the needle assembly after the striking action of FIG. 5.

FIG. 17 shows a cross-section of a first hub of a fifth embodiment of the safety syringe of FIG. 1.

FIG. 18 shows a cross-section of a second hub of a fifth embodiment of the safety syringe of FIG. 1.

FIG. 19 shows a cross-section of the safety syringe of FIG. 1 with a needle assembly according to this invention assembled using first and second hubs of FIGS. 17 and 18 with the needle assembly shown prior to retraction of the needle into the needle assembly.

FIG. 20 shows a cross-section of the safety syringe of FIG. 1 with a needle assembly according to this invention assembled using first and second hubs of FIGS. 12 and 13 modified in accordance with a sixth embodiment with the needle assembly shown prior to retraction of the needle into the needle assembly.

FIG. 21 shows an aerial view of a section of the needle assembly of FIG. 20 taken along phantom lines a and b of FIG. 20.

FIGS. 22, 23 and 24 show yet seventh, eighth and ninth needle assembly embodiments of this invention.

FIG. 25 shows yet a tenth needle assembly of this invention.

FIGS. 26 through 30 show yet further embodiments of this invention.

### DESCRIPTION OF THE PREFERRED EMBODIMENTS

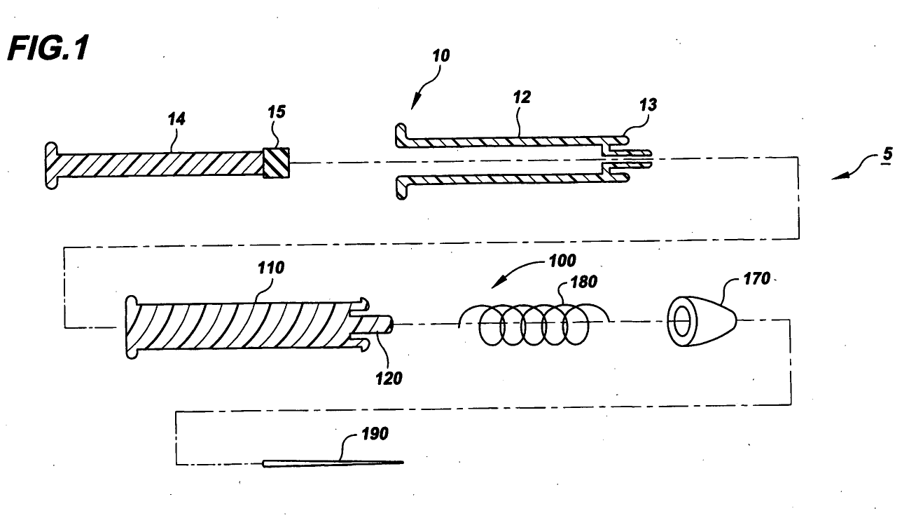

FIG. 1 shows a safety syringe 5 of this invention comprising a conventional syringe 10 and a needle assembly 100 of the present invention.

Syringe 10 comprises a barrel 12 with a Luer Loc 13 threading to allow for connection with needle assembly 100. Syringe 12 receives plunger 14 having an elastomeric head 15. Syringes of other types and construction and provided with attachments other than the Luer Lok may also be used with this invention.

In the preferred embodiment of FIG. 1, needle assembly 100 comprises a first hub 110, a second hub 170, a spring 180 disposed therebetween, and a needle 190.

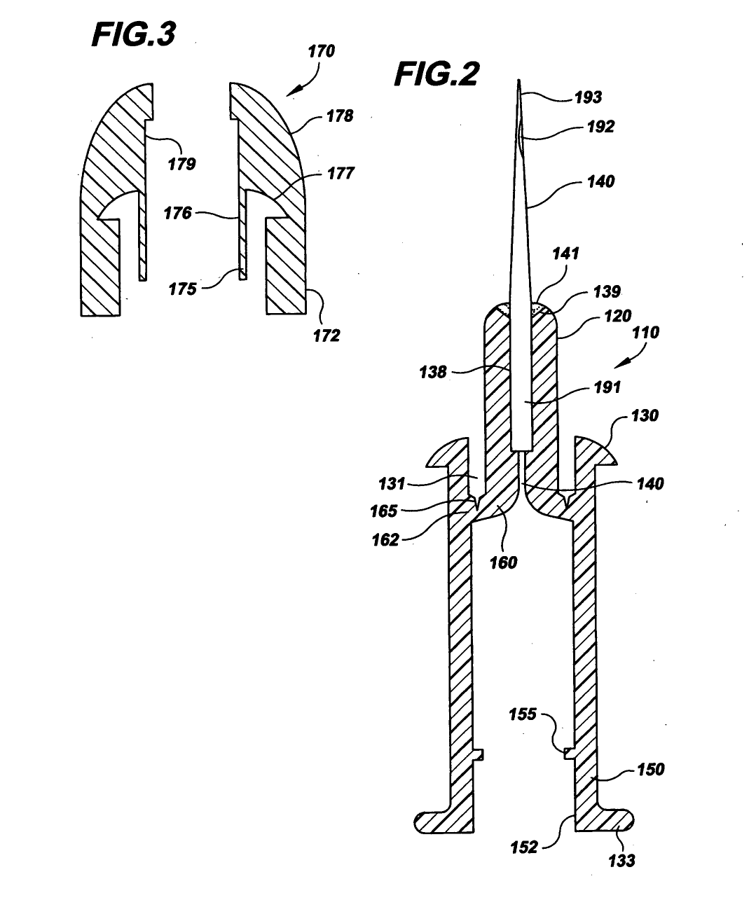

As shown in FIG. 2, first hub 110 has a first end 150, a second end 120, and a conduit 140 connecting the two. First end 150 defines a cavity 152 and a stop 155. Second end 120 of first hub 110 has an inner portion 160 and an outer portion 162, and inner portion 160 is made to be movable with respect to outer portion 162 on the application of a force.

Preferably, the inner and outer portions are an integrally molded structure that is weakened along the boundary between the two by score lines 165. Preferably, score lines 165 extend from a receptacle 131 end of second end 120 partway through the boundary between the two portions. The score lines introduce a weakness at the point of the boundary that allows inner portion 160 to break away from outer portion 162 on the application of a force.

First hub 110 defines a connector 130, the receptacle 131, a needle holder 138, a depression 139, and a connector flange 133. Connector 130 connects with a receptacle 177 defined in second hub 170. Receptacle 131 receives a protruding member 175 of second hub 170.

Needle 190 has a first end 191, a second end 193 and a lumen 192 extending therethrough. Needle holder 138 receives first end 191 of needle 190. First end 191 of needle 190 is so connected to second end 120 of first hub 110 as to allow there to be fluid communication between lumen 192 and conduit 140 of first hub 110. Depression 139 is filled with an adhesive 141 to secure needle 190 to first hub 110.

Connector flange 133 is a flange that allows first hub 110 to so mate with Luer Lok threads 13 of syringe barrel 12 as to attach first hub 110 to syringe barrel 12. Alternatively, any design that connects a needle assembly to a syringe can be used.

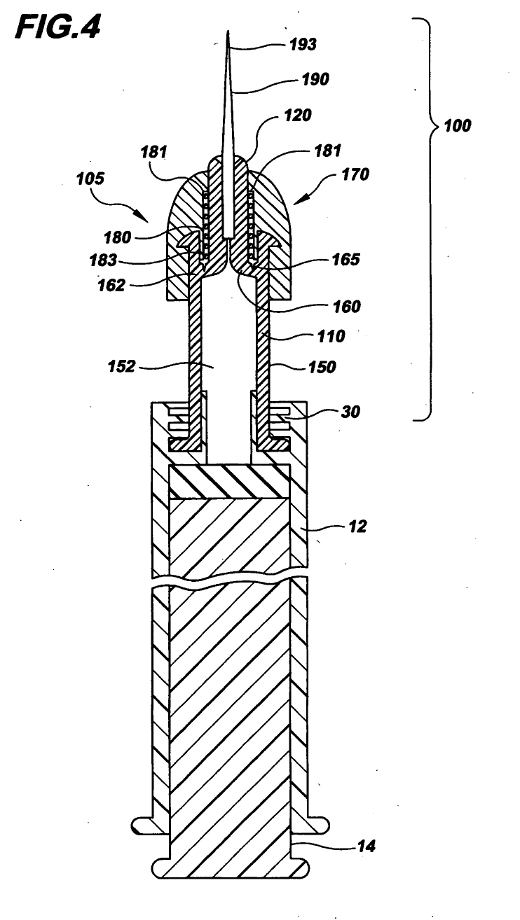

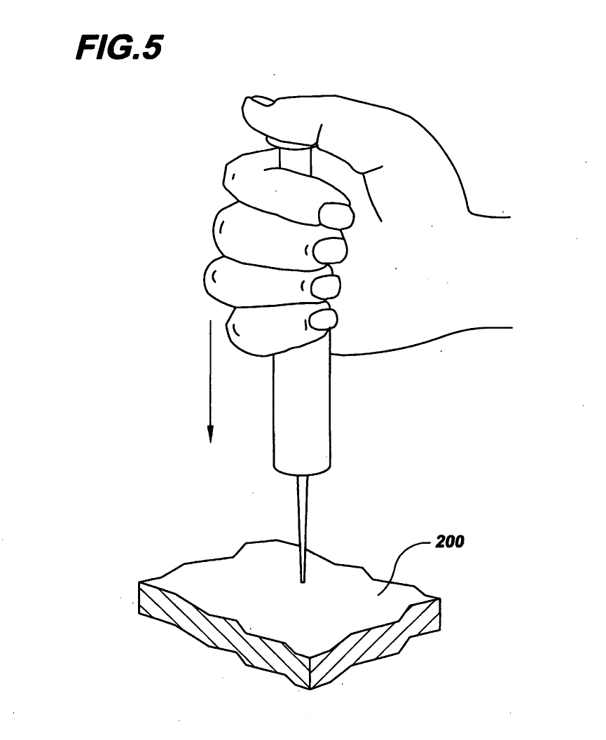

As shown in FIG. 3, second hub 170 has a first end 172, a second end 178, and a conduit 176 for receiving first hub 110. As previously indicated, receptacle 177 of second hub 170 receives connector 130 of first hub 110, and protruding member 175 of second hub 170 is received by receptacle 131 of first hub 110 when the first and second hubs are snap fitted together as shown in FIG. 4.

Spring 180 (shown in FIGS. 1 and 4) is disposed between first hub 110 and second hub 170 prior to snap fitting the two hubs together. Spring 180 is preferably a coil spring. Alternatively, spring 180 can be any spring design. It can be any material, such as elastomeric material that can provide the biasing force between first hub 110 and second hub 170. A pressurized gas appropriately sealed from the outside elements can also be used to provide the biasing force between first and second hubs, 110 and 170, respectively. As shown in FIG. 3, a stepped portion 179 of conduit 176 of second hub 170 houses spring 180 when the first and second hubs are snap fitted together. When so housed, a first and second end, 181 and 183, respectively, of spring 180 is seated against second hub 170 and inner portion 160 of first hub 110, respectively.

FIG. 4 shows a cross-section of the needle assembly of FIG. 1 assembled using first and second hubs 110 and 170, respectively, of FIGS. 2 and 3, with needle assembly 100 shown prior to retraction of needle 190 into housing 105.

Needle 190 is caused to retract into needle assembly 100 on the application of a force. In the illustrated embodiment, this force is created by the striking of second end 193 of needle 190 of needle assembly 100 shown in FIG. 4 against a strike pad 200 as shown in FIG. 5. On the application of this force, score line 165 causes inner portion 160 to break away from outer portion 162. The use of the hand in creating this force allows for a great deal of force to be brought to bear to effect the break. The break allows inner portion 160 to be moveable with respect to outer portion 162. Spring 180 urges inner portion 160 toward first end 150 of first hub 110.

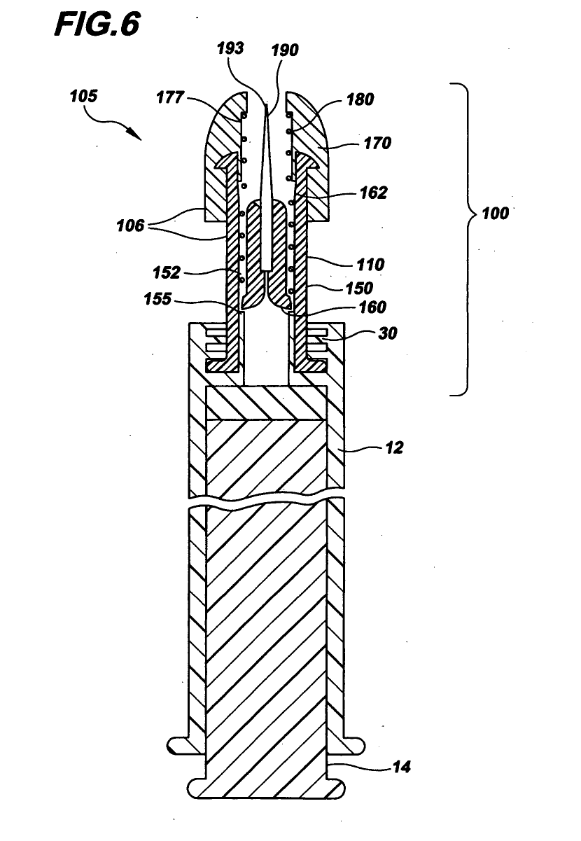

FIG. 6 shows the syringe of FIG. 4 after inner portion 160 has been broken away from outer portion 162. As shown in the figure, spring 180 has urged inner portion 160 with needle 190 deep inside cavity 152 until it is brought to rest by stop 155. During the retraction process, fluid extant inside cavity 152 prior to the needle retraction is forced to flow through a gap that exists between inner portion 160 of second end 120 of first hub 110 and the wall of cavity 152 and into the small space previously occupied by second end 120 and spring 180. Fluid tension caused by the walls defining the small space retards the leaking of fluids out of needle assembly 100. At that point, needle 190 has been entirely contained within needle assembly 100, making the syringe safe from needle strike.

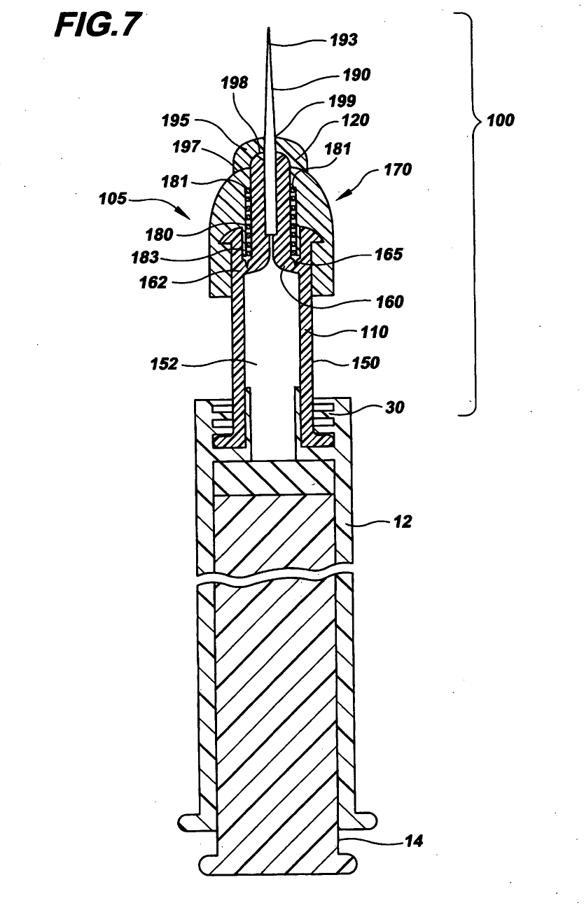

In a further embodiment shown in FIG. 7, in the needle assembly shown in FIG. 4, the combined assembly of first hub 110 and second hub 170 is provided with a collar 195 which is contoured to slip over needle 190 so as to seat preferably against second hub 170 and second end 120 of first hub 110. Collar 195 is preferable attached by an adhesive to second hub 170 only so as to allow second end 120 of first hub 110 and needle 190 to retract into needle 105 freely on the application of force to needle assembly 100. On retraction of the needle, the small opening of collar 195 further prevents fluid from leaking out of needle assembly 100.

Preferably, collar 195 is a rubber or other material having an elastic property that allows the material to grip the needle yet allow the needle to retract when acted on by a force. This allows the needle to bias or “stretch” the collar 195 outwardly such that on retraction, the collar 195 relaxes into the space left behind the retracted needle to prevent fluid leakage. Alternatively, any rubber, elastomer, plastic or other material that may but need not touch or grip needle 190 and that provides some cover across the opening left behind by the structure that retracts with the needle can be used. These are some illustrative ways in which fluid leakage may be further prevented.

In the foregoing embodiments, the needle assembly 100 comprises second hub 170 defining a conduit (conduit 176 in this case), first hub 110 having inner portion 160 (made to be moveable with respect to outer portion 162 on the application of a force), spring 180 disposed therebetween, and needle 190. It will be appreciated, however, with reference to FIG. 6, for example, that more broadly, needle assembly 100 of the preferred embodiment comprises: housing 105 having a first part 106 (comprising second hub 170 and first end 150 of first hub 110) and a second part 116 (comprising second end 120 of first hub 110), and spring 180. First part 106 defines the previously disclosed cavity 152 and second part 116 moves with respect to first part 106 into said cavity to effect the shielding by housing 105 of needle 190 against needle strike. Alternatively, first part 106 defines a conduit in which second part 116 moves in retraction to make the needle safe.

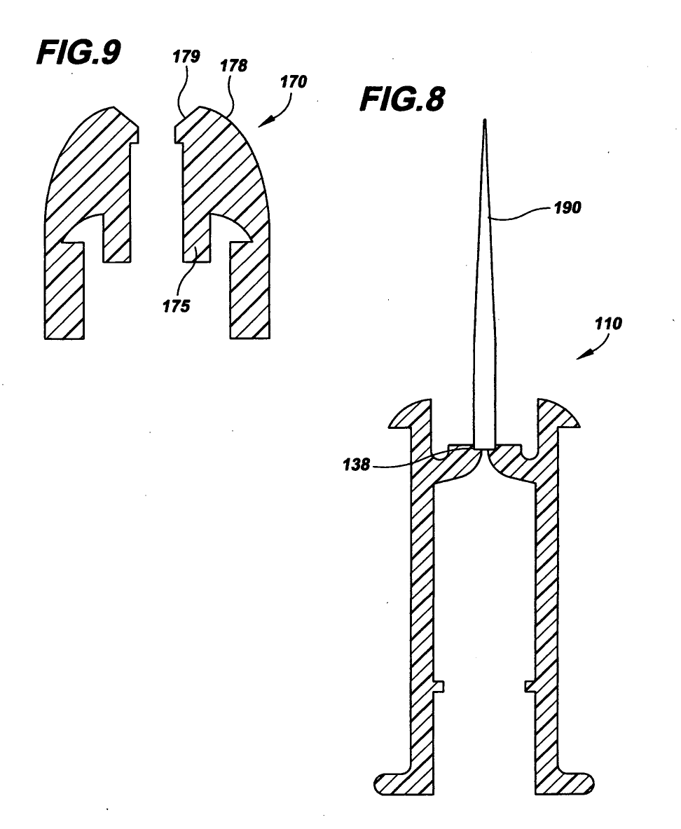

FIG. 8 shows a cross section of a first hub 110 of a third embodiment of the needle assembly of FIG. 1. FIG. 9 shows a cross section of a second hub 170 of the third embodiment of the needle assembly of FIG. 1.

FIGS. 8 and 9 contain structural features similar to those in the first and second hubs shown in FIGS. 2 and 3, although there are some differences. Needle holder 138 of first hub 110 shown in FIG. 8 is shorter than needle holder 138 of FIG. 2. Protruding member 175 of second hub shown in FIG. 9 is made to be thicker than protruding member 175 of FIG. 3. In addition, first end 178 of second hub 170 shown in FIG. 9 is also provide with an angled surface 179.

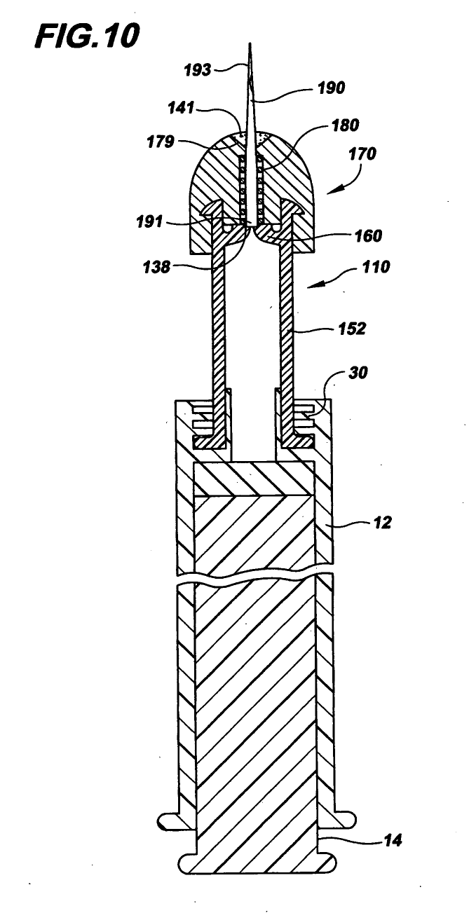

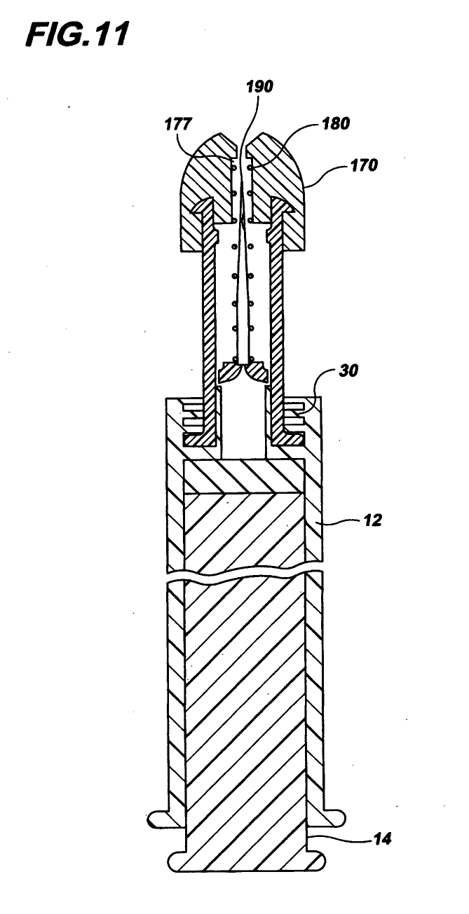

FIG. 10 shows the safety syringe of FIG. 1 with a needle assembly of this invention assembled using first and second hubs of FIGS. 8 and 9. As shown in FIG. 10, needle 190 is held to first hub 110 at its first end 191 by needle holder 138 defined in first hub 110. At its second end 193, needle 190 is held in place by adhesive 141 which sits in a depression created by angled surface 179. The lesser structure around needle 190 allows for cavity 152 to define a smaller volume for receiving fluid when compared to the needle assembly shown in FIG. 4. The smaller cavity results in less fluid moving through the gap between the first portion 160 of first hub 110 and the wall of cavity 152 during the needle retraction process. Less fluid passing through the gap can reduce the resistance of the fluid to the retraction process, reduce fluid leakage, and reduce the amount of unspent fluids, such as a medication, retained in the needle assembly. FIG. 11 shows the safety syringe of FIG. 10 after the needle has been retracted and made safe.

It will be appreciated from this invention that the volume to be defined by the cavity of the inventive needle assembly should preferably be not much greater than the volume displaced by the needle when retracted. In the earlier-described embodiments, the volume of the cavity was defined substantially by the length of the needle times the greatest cross-sectional area of the inner portion 160 that needs to traverse the cavity. As the length of the needle becomes more uniform (standard), by reducing the cross-sectional area of inner portion 160, the volume defined by the cavity for receiving the retracted needle can be further minimized.

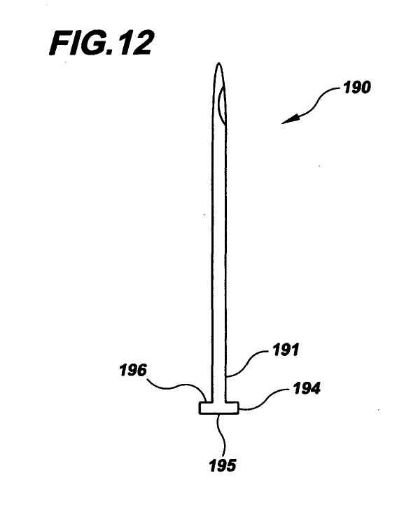

FIG. 12 discloses an inventive needle 190 for minimizing the cross-sectional area of the inner portion 160 that needs to traverse the cavity. Inventive needle 190 comprises a stop 194 at first end 191 of needle 190. Preferably stop 194 and needle 190 are formed as an integrated structure from the same material. Alternatively, stop 194 can be formed from a material separate from first end 191 of needle 190 and held together with first end 191 to form needle 190 by use of, for example, an adhesive, friction fit, solder or other bond. Preferably, as an integrated structure made from the same material as needle 190, stop 194 has a hardness that breaks-away from outer portion 162 quickly and leaves a cleaner break. As a result, the area defined by face 195 of needle 190 times the length of the needle can define a volume for the cavity of an even smaller size.

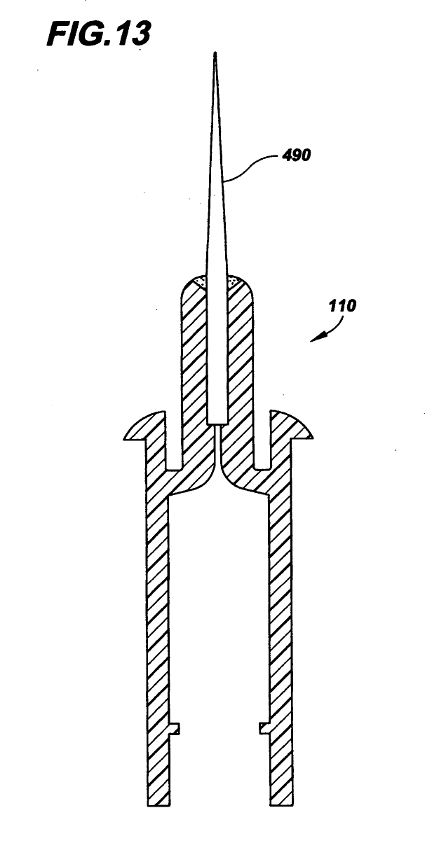

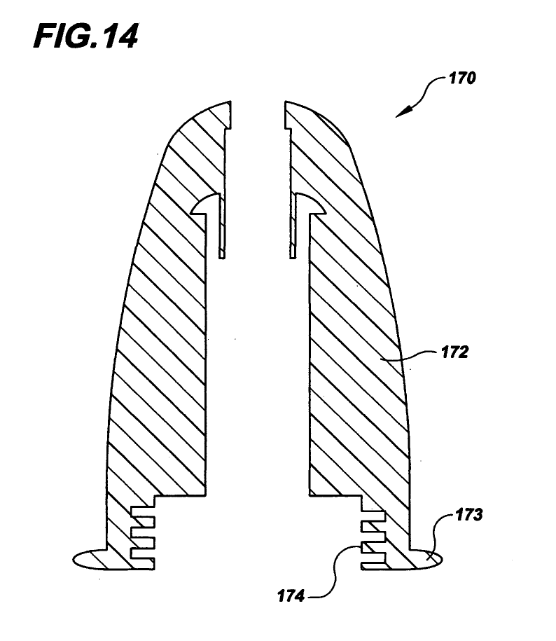

FIG. 13 shows a cross section of a first hub of a fourth embodiment of the needle assembly of FIG. 1. FIG. 14 shows a cross section of a second hub of the fourth embodiment of the needle assembly of FIG. 1.

FIGS. 13 and 14 contain several structural features similar to those contained in the first and second hubs shown in FIGS. 2 and 3, although there are some differences. Flange 133 defined in hub 110 shown in FIG. 2 is missing from hub 110 shown in FIG. 13. First end 172 of second hub 170 shown in FIG. 3 is elongated. In addition, in FIG. 14, Luer Lok threads 174 are provided along an inside surface of second hub 170 and a finger flange 173 is also provided to allow needle assembly 100 to be easily attached to a syringe.

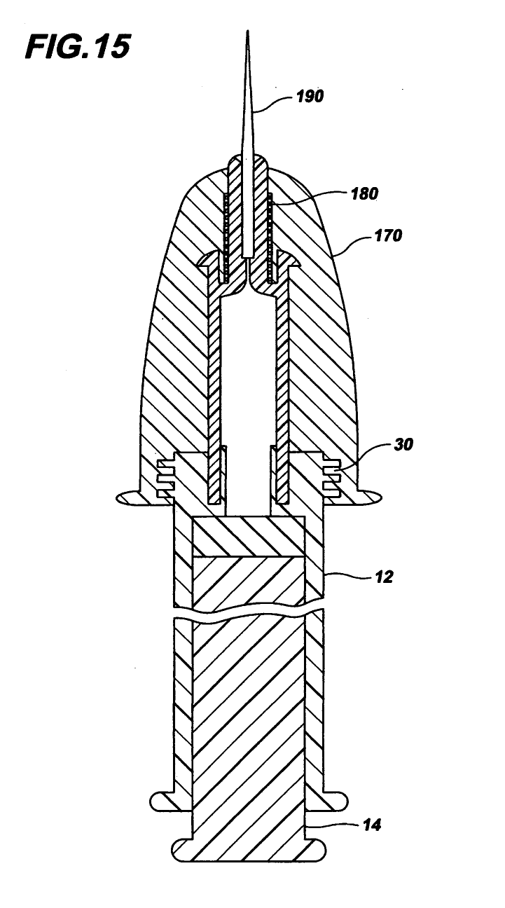

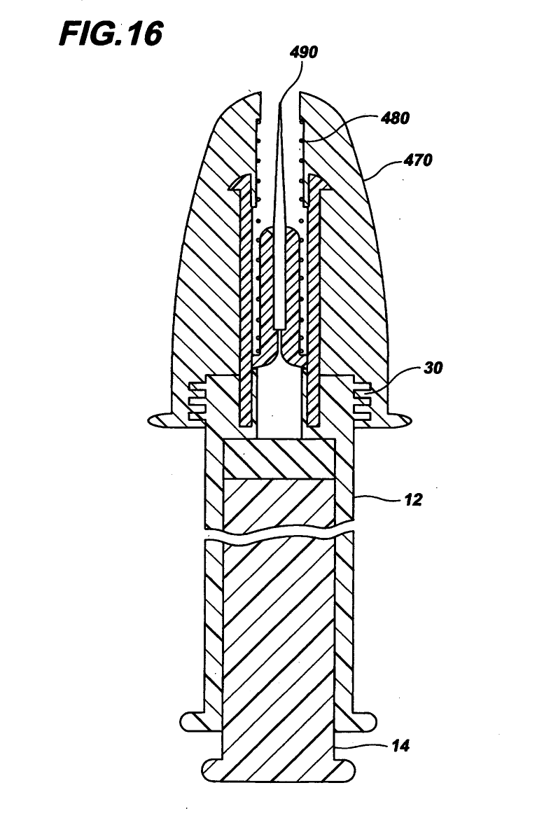

FIG. 15 shows the safety syringe of FIG. 1 with a needle assembly of this invention assembled using first and second hubs of FIGS. 13 and 14. FIG. 16 shows the safety syringe of FIG. 15 after the needle has come to rest inside the needle assembly.

In at least one embodiment described earlier, inner and outer portions, 160 and 162, respectively, of first hub 110 were made as an integrated structure. Score lines were used to introduce a weakness at the boundary between inner and outer portions, 160 and 162, respectively, so as to allow inner portion 160 to break away from outer portion 162 on the application of a force. It will be appreciated that the boundary between inner and outer portions may be weakened using techniques for structures other than score lines so as to allow the inner and outer portions to move with respect to each other. For instance, at the boundary between the two portions, recesses may be provided to so weaken the boundary between the two as to allow inner and outer portions to separate and move with respect to each other on the application of a suitable force. Preferably, the one or more recesses may be provided on the side of the boundary facing away from the fluid side although recesses on the fluid side or on both sides can also be used. As still another example, discontinuities can be introduced into the structure such as at or near the boundary. For instance, brittle material could be used at or near the boundary that would easily break on application of a suitable force. Pores can be introduced at or near the boundary zone or region that will also break more easily on the application of a suitable force. The boundary zone between the inner and outer portions can be made to be thinner than the surrounding portions so as to break on the application of an appropriate force. The inner or outer or both portions can also be dimensioned to break at or near the boundary zone. Projections may be defined into the structure so as to allow the inner and outer portion to break away from each other so as to allow each to move with respect to the other. The outer and/or inner portion at the boundary zone may have defined into its structure notched sheer pins or sheer pins with stress risers that allow for separation and movement between the two portions.

It will also be appreciated that first and second portions, 160 and 162, respectively, may also be formed from two separate materials and held together with a breakable adhesive. Alternatively, the two materials may be held together by a friction fit. One or more projections on one of the two portions may so interconnect with one or more openings or receivers on the other portion as to provide a connective fit between the two, by snap fit or other connection, for example, that allows the two portions to become movable on the application of an appropriate force.

EP1-924443A2 and PCT WO 00/37131 describe a variety of techniques known in the art for creating a structure having portions that move with respect to one another on the application of a force, and those publications are hereby incorporated by reference.

In the preferred embodiment, a striking of the needle against a strike pad as shown in FIG. 5 is the force applied to the inventive needle assembly used to effect a retraction of the needle into the needle assembly. It will be appreciated that other forces and techniques may be used to effect this retraction.

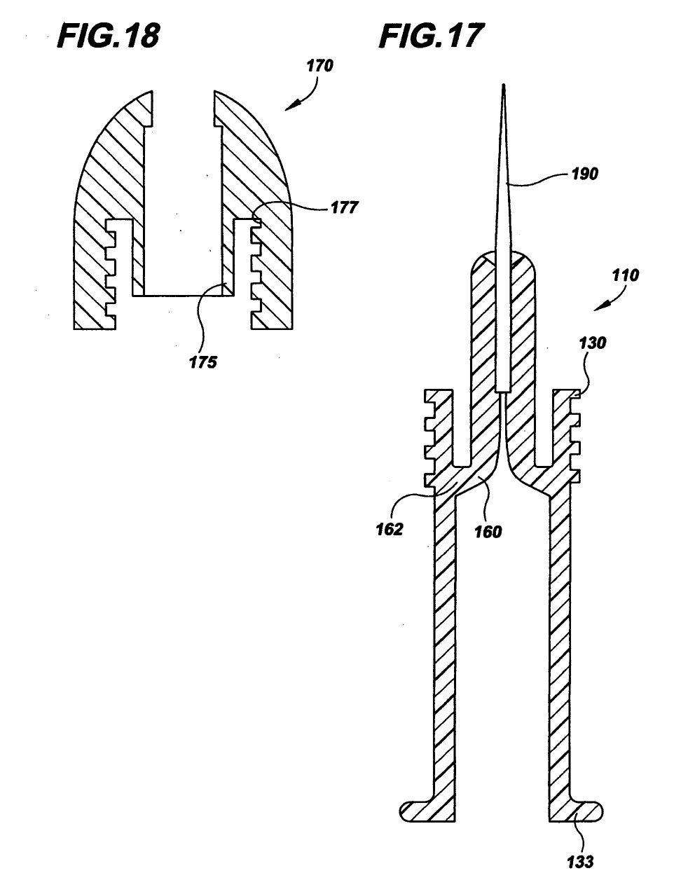

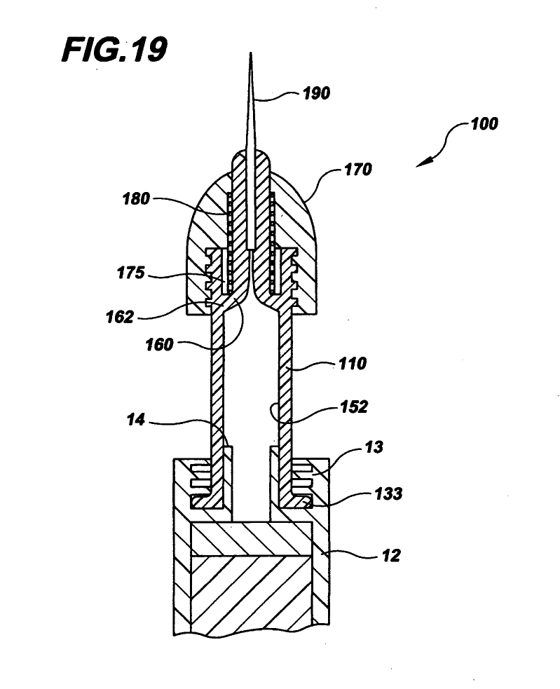

The force applied may, for instance, be a rotational force. FIG. 19 shows such an embodiment of the safety syringe of FIG. 1 with a needle assembly of this invention assembled using first and second hubs, 110 and 170, respectively, of FIGS. 17 and 18. FIGS. 17 and 18 contain structural features similar to those in the first and second hubs shown in FIGS. 2 and 3 although there are some differences. As shown in FIG. 17, first hub 110 is provided with a threaded collar 130 which threadingly engages threads 177 provided along an inside wall of second hub 170. Attachment of first and second hubs is by this threading engagement. Attachment of needle assembly 100 to syringe barrel 12 is by threading engagement of connector flange 133 with Luer Loc 13.

On further rotation of second hub 170 about first hub 110, protruding member 175 of second hub 170 breaks the boundary zone between inner and outer portions, 160 and 162, respectively. Inner portion 160 with needle 190 is then retracted into cavity 152 of needle assembly 100 under the influence of spring 180.

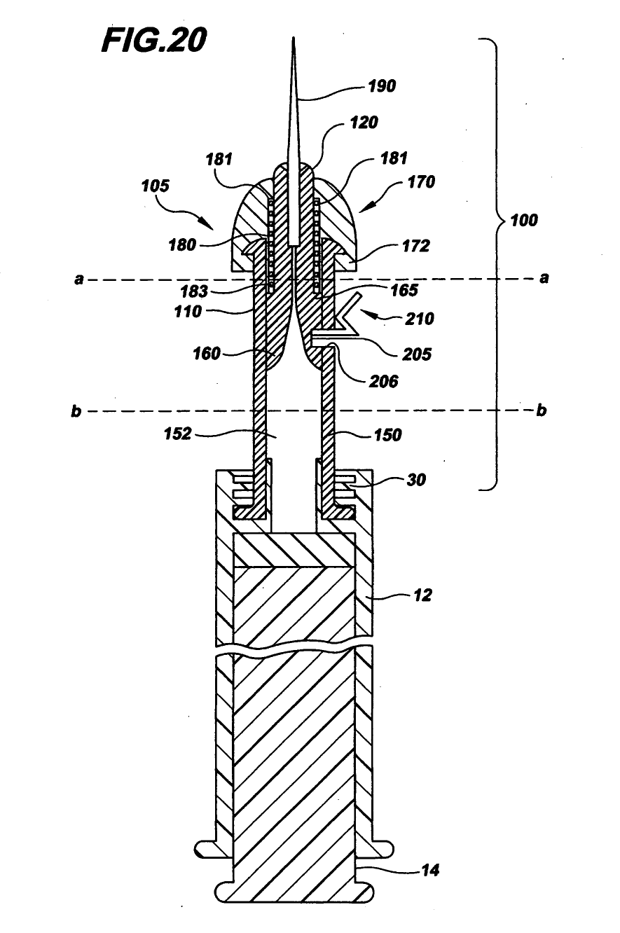

The force may also be a translational force applied by, for example, activation of an activation button as shown in yet a sixth embodiment shown in FIG. 20. Needle assembly 100, shown attached to syringe barrel 12, comprises a first hub 110 and second hub 170 which contain several structural features similar to those in the first and second hubs shown in FIGS. 2 and 3, although there are some differences. Second hub 170 is so modified at first end 172 as be shorter than first end 172 shown in FIG. 3. First hub 110 is also modified as shown in FIGS. 19 and 20. Section 120 of first hub 110 has inner portion 160 but no outer portion. Inner portion 160 is slideably and sealingly engaged against cavity 150 and extends deeper into cavity 150 than inner portion shown in FIG. 2. A recess 205 is defined lengthwise along an outer surface of the extended portion of inner portion 160. A slot 206 is defined lengthwise in first end 150 of first hub 110. Slot 206 is placed into alignment with recess 205 during assembly.

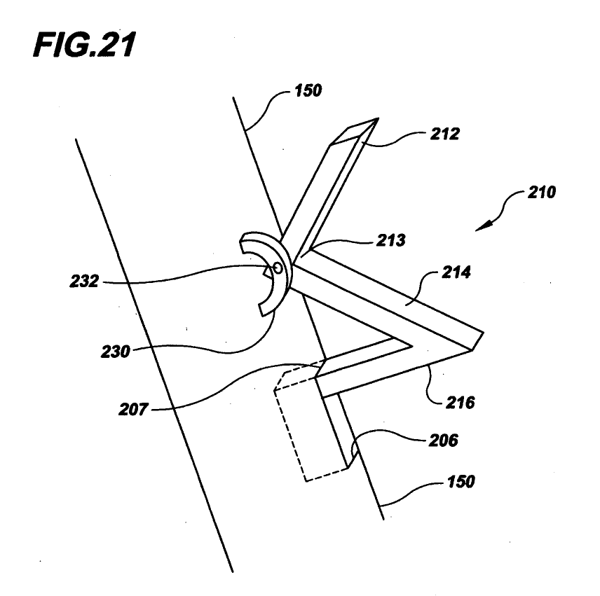

As shown in FIG. 21, needle assembly 100 further comprises a rocker arm 210 comprising a stop section 216, a mid section 214, a connector section 213, and a lever arm 212. First hub 110 is provided with an outwardly extending connecter 230 which receives connector section 213 of rocker arm 210. A pin 232 holds connector section 213 of rocker arm 210 to outwardly extending connector 230.

As shown in FIGS. 20 and 21, when assembled, stop section 216 of rocker arm 210 is received by slot 206 and recess 205. Spring 180 so biases first hub 110 that stop section 216 is held against stop section 207 of slot 206 and recess 205. Retraction of needle 190 into cavity 150 occurs on-activation of lever arm 212 which causes rocker arm 210 to rock such that stop 216 disengages from recess 205. Without stop 216 opposing the bias of spring 180, member 120 with needle 190 is urged into cavity 150 by spring 180. Containment of needle 190 inside needle assembly 100 protects against needle strike.

As shown in a further embodiment disclosed below, the force applied to retract the needle may also be a force applied to the plunger of the syringe.

It will be appreciated that the “stop” used to bring the needle to a rest inside the needle assembly is preferably a structure defined by the needle housing. Alternatively, the stop used to bring the needle to a rest inside the needle assembly may be structure defined by the syringe. Referring again to FIGS. 17, 18 and 19, one of the differences additional to what was disclosed above between the FIGS. 17 and 18 hubs and the first and second hubs shown in FIGS. 2 and 3 is the structure used to bring the needle to a rest inside the needle assembly. In the first hub of FIG. 2, first hub defines a stop 155 and the needle is brought to a rest inside the needle assembly by the stop 155. See FIG. 6 showing the assembled FIGS. 2 and 3 hubs after the needle has been retracted. The hub of FIG. 17 is without such a stop. In the FIG. 19 embodiment showing the assembled FIGS. 17 and 18 hubs, the structure used to “stop” the needle inside the needle assembly is not a structure defined by needle assembly 100. Rather, the structure used to “stop” the needle inside the needle assembly is lip 14 of barrel 12 of syringe 10.

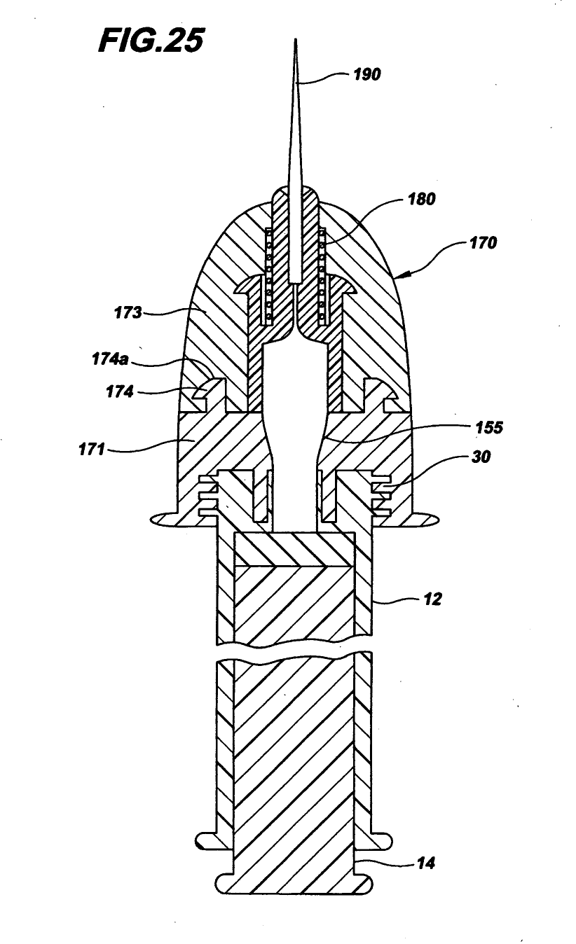

It will be further appreciated that in embodiments comprising a first and a second hub, that just as stop 155 is defined by the first hub, so too stop 155 can be defined by the second hub. FIG. 25 shows such a needle assembly wherein stop 155 is defined by second hub 170. In this embodiment, second hub 170 comprises a first member 171 and a second member 173. Preferably, first member 171 further defines a connector 174 and second member 173 defines a receptacle 174a for receiving connector 174 in a “snap fit”. Alternatively, first and second members, 171 and 173, respectively, may be held together by an adhesive, friction or a connective fit. Any stop defined by the needle assembly or syringe that brings the needle to a rest inside of the needle assembly after the needle has been retracted is within the scope of this invention.

The shape or form of the structure used to bring the needle to a rest inside the needle assembly is less important than that the structure brings the needle to a rest inside the needle assembly. Consequently, the invention is not limited to the shape or form of the “stops” disclosed in the embodiments. The “stop” may be an annular ledge that circumscribes the inner wall of cavity 152 of first hub 170, for example. It may be one or more ledges or projections that protrude inwardly from inner wall of cavity 152 of first hub 110, for example. It may be one or more ledges or projections that extend inwardly from the inner wall of second hub 170 of the needle assembly shown in FIG. 25, for example. It may be an inwardly tapered sidewall, such as the stop 155 shown in FIG. 25 that causes the needle to become lodged in the tapered section of the sidewall, thereby bringing the needle to a rest.

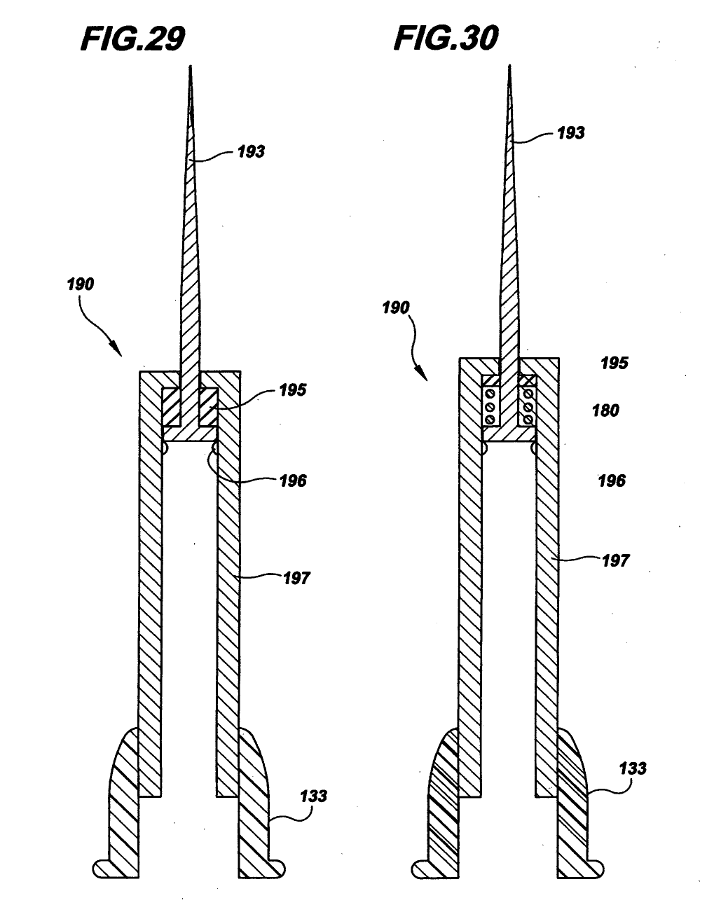

It will be further appreciated that a “stop” is not limited to a structure that stands directly in the path of the retracting needle. Any structure that prevents the needle from further retracting is within the scope of this invention. For instance, it can be a collar 195 such as shown in FIG. 7 that frictionally engages needle 190 throughout the retraction process. In this embodiment, on cessation of force, needle point end 193 is so withdrawn into collar 195 as to be protected by collar 195 against accidental needle strike and yet so continued to be frictionally held or “stopped” by collar 195 as to be prevented from further retracting. In another embodiment, a thicker collar 195 is used to allow more protective and frictional action to be brought to bear on the needle and point during the retraction process for this purpose. While collar 195 shown in FIG. 7 is seated outside of cavity 152, a collar or gasket 195 seated inside of cavity 192 as shown in FIG. 29 herein described is also within the scope of this invention.

In another embodiment, the walls or other structure defined by cavity 152, for example, of FIG. 6, for example, may frictionally co-act with the retracting needle to “stop” the needle from further retraction after needle point end 193 has been protected. For instance, and referring to FIG. 2, the walls defined by cavity 152 may be smoother nearer second end 120 of first hub 110 than they are nearer first end 150 of first hub 110. During retraction, the smoother walls acting against inner portion 160 allow needle 190 to retract while the less smoother walls co-act with inner portion 160, as the needle is retracting, to bring needle 180 to rest. Alternatively, walls defined by cavity 152 may provide a more uniform frictional co-action with inner portion 160 which is insufficient to resist the initial action of spring 180 when the needle is retracting yet sufficient to resist later action of spring 180. In yet another embodiment, spring 180 does not retract the needle the full length of the cavity. Consequently, when spring 180 has retracted needle 190 as far as it can, there is still some cavity for the needle to travel through which sidewalls frictionally co-act with inner portion 160 to bring needle 190 to rest.

While “stops” defined by structure have been illustrated, it will be appreciated that non-structural “stops” are also within the scope of this invention. For instance, and referring to FIG. 7, retraction of the needle causes unspent fluid in cavity 152 to flow around inner portion 162 into the space previously occupied by second end 120 and spring 180. When there is no more space for unspent fluid to flow into, the remaining fluid will resist any further retraction of the needle. At this point, the resistance to the retracted needle of unspent fluids remaining in the lower portion of cavity 152 acts to “stop” the needle from further retraction.

It will also be appreciated that this invention is directed to a safety syringe that so shields the needle as to prevent accidental needle strike when the invention is not being used. Protection of the point of needle strike—namely, the needle point, that is, for example, second end 193 shown in FIG. 2)—by the needle assembly is an important feature of this invention.

Preferably, the entire needle will come to rest inside the needle assembly after the retraction. In an alternative embodiment, the needle assembly protects the needle point after retraction while allowing some or the rest of the needle to retract partway into the syringe. So long as the needle assembly is protecting the needle point after the retraction, it falls within scope of this embodiment of the invention. Technical implementations for retracting a needle into a syringe from a needle assembly are known in the art. See, for example, EP1-924443A2 and PCT WO 00/37131, which are incorporated herein by reference. In those prior art, the needle and/or supporting structure travels into a cavity formed in the plunger that penetrates the bore of the syringe. In this alternative embodiment, those prior art devices are so modified as to shorten the length of travel that the needle and/or supporting structure can travel into the cavity formed in the plunger. When, after a retraction, the needle and/or supporting structure reaches the end of the cavity or the “stop” in this embodiment, the needle point comes to rest inside of and is protected by the inventive needle assembly of this invention notwithstanding other portions of the needle residing in the syringe. This embodiment further illustrates that the force that can be applied to retract the needle can be one applied to the syringe, such as in this example, the plunger of the syringe.

Any stop, whether structure or other “stop” defined by or in the needle assembly or syringe or syringe device or accessory adapted to either, that acts to bring the needle to a rest inside the needle assembly after the needle has been retracted is within the scope of the present invention.

In the previous embodiments, the needle assembly comprises: a housing having a first part and a second part, said first part defining a cavity therein and said second part being movable with respect to said first part; a needle attached to said second part; a spring located between said first part and said second part; wherein on application of a force, said second part moves with respect to said first part allowing said spring to retract the needle into said cavity defined by said first part of said housing; and wherein, after retraction, said needle comes to a rest inside said cavity, said housing shielding said needle against needle strike.

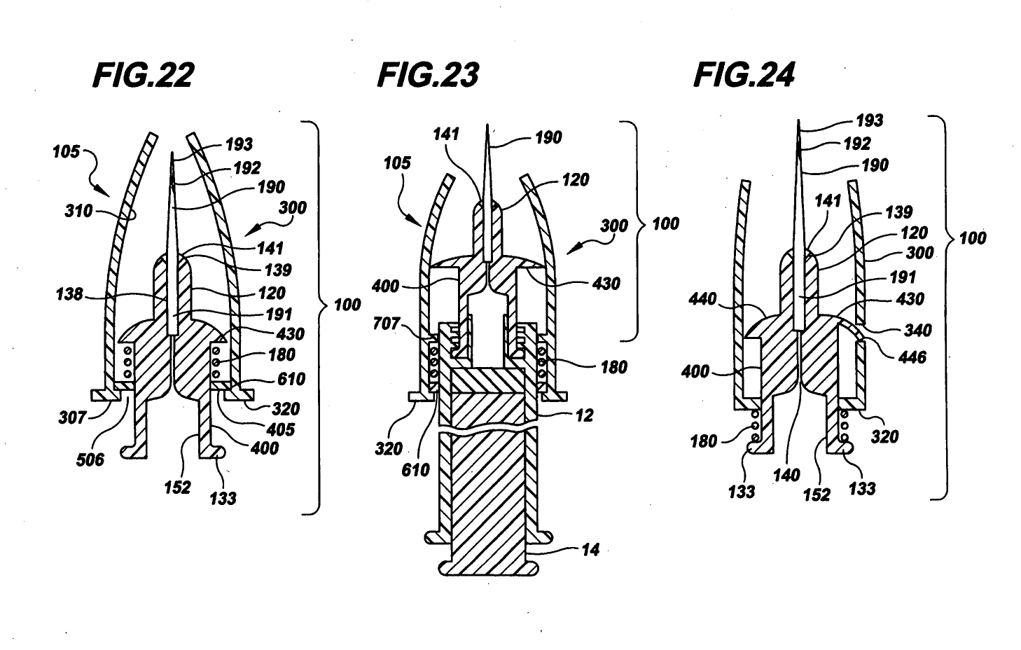

In an alternative embodiment, as shown in FIG. 22, needle assembly 100 comprises: a housing 105 having a first part 300 and a second part 400, said first part 300 defining a conduit 310 therein and being movable with respect to said second part 400; a needle 190 attached to said second part; a spring 180 located between said first part 300 and said second part 400; wherein on application of a force, said first part 300 moves with respect to said second part 400 allowing said spring 180 to so bias first part 300 as to shield needle 180 by said conduit 310 defined by said first part 300 of said housing 105; and wherein, after so biasing the first part 300, said needle 190 sits inside said conduit 310, said housing 105 shielding said needle 190 against needle strike.

In FIG. 22, some of the structural elements are similar to corresponding elements shown in FIG. 4 with some modifications. Needle 190 is received by a second end 120 of second part 400. An adhesive 141 deposited in depression 139 secures needle 190 to second part 400.

In addition, first part 300 defines a stop 320. Spring 180 is seated between a stop surface 307 of stop 320 of first part 300 and a stop 430 of second part 400. Second part 400 has an outwardly tapered sidewall 405 and a gap 506 extends between tapered sidewall 405 and stop 320. Gap 506 allows stop 320 to “clear” sidewall 405 as first part 300 is retracted.

Needle assembly 100 further comprises a gasket 610 which is designed to tightly frictionally engage shield 300 and further to less tightly frictionally engage outer wall 405 of second part 400 tighter and tighter as first part 300 is retracted to expose needle 190. Preferably, gasket 610 is made from a rubber so as to elastically stretch during the retraction process so as to frictionally engage outer wall 405. Alternatively, gasket 610 can be made from an elastomer or other material having elasticity and/or frictional properties necessary to keep second part 400 so retracted as to expose needle 190 during use. In this embodiment, over time, the stretching properties of the rubber used causes gasket 610 to so “give” as to its frictional holding of outer wall 405 as to cause it to slip along wall 405. When slippage occurs, first part 300 is urged by spring 190 back into the shield position.

A connector flange 133 allows needle assembly 100 to mate with Luer Lok threads of a syringe barrel (not shown).

In the FIG. 22 embodiment, the distance between stops 320 and 430 defines one distance of retraction. As this distance is lengthened, a longer shield can be used to protect the needle in the protect position.

FIG. 23 shows another needle assembly of this invention. The needle assembly is shown in the retracted position with first part 300 so modified as to extend over syringe barrel 12. A gasket 610 frictionally engages stop 320 to syringe barrel 12. Spring 180 sits between gasket 610 and a stop 707 inside the cavity defined by first part 300.

The embodiment is shown in the retracted position with the spring energized by the retraction. Relaxation of first part 300 by manually sliding stop 320 of first part 300 forwardly of gasket 610 allows spring 180 to urge stop 707 against a stop 430 formed in second part 400. At this point, first part 300 extends forwardly of needle 190 so as to protect it.

In still another embodiment shown in FIG. 24, a needle assembly 100 of the invention is shown provided in a retracted position. Spring 180 is disposed between stop 320 and connector flange 133, which is used to connect needle assembly 100 to Luer Lok threading of a syringe barrel (not shown). A slot 340 formed lengthwise along first part 300 receives a leaf spring 446 extending from second part 400. At other points along the circumference of second part 400 on which leaf spring 446 is seated is formed a stop 440. During retraction, first part 300 urges spring 180 against stop 133 causing spring 180 to energize. First part 300 slips over leaf spring 446 during this retraction process until leaf spring 446 slips through slot 340. At this point, leaf spring 446 holds first part 300 in the retracted or “cocked” position with respect to second part 400 until such time as leaf spring 446 is depressed. When that occurs, first part 300 is urged forward to protect needle 190.

While a leaf spring is taught in this embodiment as a mechanism for keeping first part 300 “cocked”, it will be appreciated by those skilled in the art that other mechanisms for keeping a shield in the retracted position known in the art may be used with this invention and are hereby incorporated by reference.

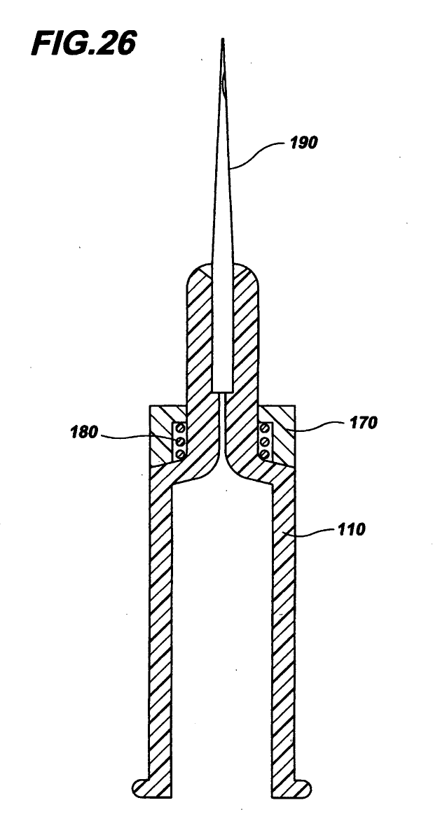

FIG. 26 shows a further embodiment of this invention which contains structural features similar to those in FIGS. 2 and 3, although there are some differences. In this embodiment, second hub 170 is connected to first hub 110 with an adhesive although a snap fit connection of the kind shown in FIG. 4 may also be used. The lip of the syringe (not shown) used with this embodiment provides the “stop” in this example.

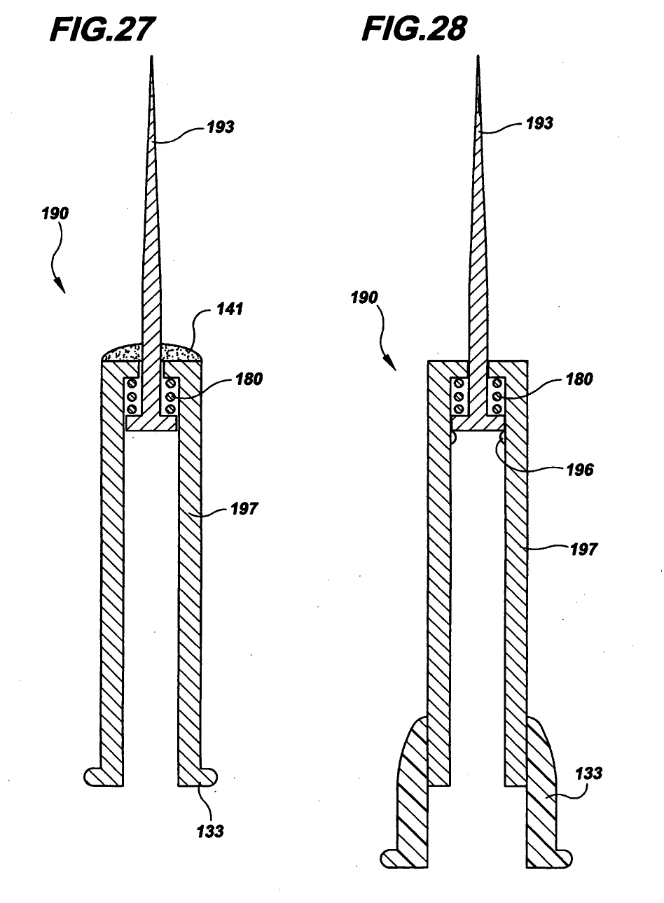

FIG. 27 shows a retractable needle 190 of this invention comprising a needle 193 of the kind disclosed in FIG. 12, needle housing 197, and spring 180. As shown, spring 180 is preferably disposed between needle 193 and needle housing 197 and adhesive 141 holds needle 193 to needle housing 197 in a “cocked” position. Needle housing 197 is preferably made from a metal of the type used to make needle 193, although plastic, other metal or other material may be used to make housing 197. A connector 133 allows needle housing 197 with needle 193 to be connected to a syringe. The retraction of needle 193 is “stopped” by the lip of the syringe (not shown) with which this embodiment is used.

FIG. 28 shows yet another embodiment of a retractable needle of this invention and contains structural features similar to those in the FIG. 27, although there are some differences. A gasket 196 is used to hold needle 193 to needle housing 197 in a “cocked” position. The gasket is preferably a rubber or elastomer or other material with some “stretch” properties. Alternatively, any material that sufficiently grips the inside walls of needle housing 197 so as to keep the needle 193 in a “cocked” position yet allows needle 193 to be retracted on the application of a force is within the scope of this invention. In one example, the force causes needle 193 to “squeeze” through gasket 196 with the gasket remaining in place after this happens. In another example, the force causes needle 193 to “break” gasket 196 so as to allow the retraction.

FIG. 29 shows still another embodiment of this invention containing structural features similar to FIG. 28, although there are some differences. There is no spring in this embodiment. Needle 190 further defines a gasket 195. Preferably, gasket 195 is a rubber or elastomer or other material having a “gripping” action on needle 193. In this embodiment, on cessation of a force, such as the striking of needle 193 against a surface, needle point end 193 is so withdrawn into gasket 195 as to be protected by housing 197 against accidental needle strike and yet so continued to be “grippingly” held or “stopped” by gasket 195 as to be prevented from further retracting.

FIG. 30 shows yet another embodiment of this invention containing structural features similar to FIG. 29, although there are some differences. Needle housing 197 is made from a plastic. Alternatively, any metal or other material sufficient for this purpose can be used. In this embodiment, needle 193 is a plastic of sufficient hardness to provide the desired needle function. Alternatively, needle 193 can be a metal or other material of suitable hardness. Needle 190 further comprises a spring 180 which causes needle 193 to retract on the application of a force.

While FIGS. 27-30 disclose a retractable needle wherein needle housing 197 is part of the inventive needle, in further embodiments, needle housing 197 in these embodiments forms the very “housing” for holding the needle, not unlike the “housing” for holding the needle disclosed elsewhere in this specification.

In view of the disclosure herein made, it will be appreciated that in several embodiments more broadly needle assembly comprises: a housing; a needle attached to said housing; a spring connected to said housing for moving said needle; wherein on application of a force, said spring retracts said needle point into said housing; and wherein after retraction, said needle point comes to rest inside said housing. Said application of said force being, for example by striking of the head of said needle against a surface, such as against a strike pad; by applying a rotational force to said housing; by activation of a release mechanism provided on said needle assembly; by applying a force to a syringe, such as to a plunger; or by applying some other force to either or both needle assembly and syringe.

In view of the disclosure herein made, it will be further appreciated that the foregoing housing may further comprise a first part and a second part, said second part being movable with respect to said first part, said needle point being connected to said movable second part of said housing. The first part may be configured in a variety of ways to protect the needle against a strike. For example, said first part can define a cavity where on application of said force, said needle retracts into said cavity. Alternatively, said first part can define a conduit where on application of said force, said needle retracts into said conduit.

In view of the disclosure herein made, it will be still further appreciated that when the inventive needle assembly is attached to a syringe, the combination forms an inventive syringe device. Whereupon on application of a force to said syringe device, said needle point is retracted into said housing; and wherein after retraction, said needle point comes to rest inside said housing. Said application of said force being, for example by striking of the head of said needle against a surface, such as against a strike pad; by applying a rotational force to said syringe device; by activation of a release mechanism provided on said syringe device; by applying a force to a syringe, such as to a plunger; or by applying some other force to either or both needle assembly and syringe.

In view of the disclosure herein made it will be yet further appreciated that a novel method for preventing needle strike is herein disclosed, said method comprising the steps of: attaching a needle assembly having a needle to a syringe to form a syringe device; performing an operation with said syringe device; applying a force to said syringe device to retract said needle point into said needle assembly, said needle point coming to a rest inside said needle assembly. Said step of applying a force can be by striking of the head of said needle against a surface, such as against a strike pad; by applying a rotational force to said syringe device; by activation of a release mechanism provided on said syringe device; by applying a force to a syringe, such as to a plunger; or by applying some other force to either or both needle assembly and syringe. So too appreciated will be the novel method for making the inventions herein disclosed.

It will be further appreciated that a housing that retracts and protects the needle by the needle assembly enjoys the above and other features as herein disclosed and as will be appreciated from the disclosures herein made. So too will its combination with a syringe as will it method of use and method of manufacture.

It will be further appreciated that a housing and a needle together can form a retractable needle, enjoying the above and other features as herein disclosed and as will be appreciated from the disclosures herein made. So too will its combination with a syringe as will it method of use and method of manufacture.

Further disclosed embodiments include, but are not limited, to the following:
1. A needle assembly comprising:
   a housing;
   a needle attached to said housing, said needle having a needle point;
   wherein on application of a force, said needle point is retracted into said housing; and
   wherein after retraction, said needle point comes to rest inside said housing.
2. The needle assembly of claim 1 wherein said housing and said needle together form a retractable needle.
3. The needle assembly of claim 1 wherein said needle assembly further comprises a gasket around said needle, wherein on application of said force, said needle point retracts into said gasket, said gasket protecting said needle point and preventing said needle against further retraction.
4. The needle assembly of claim 1 further comprising a spring connected to said housing for moving said needle, wherein on application of said force, said spring retracts said needle point into said housing.
5. The needle assembly of claim 4 wherein said application of force is a striking of the head of said needle against a surface.
6. The needle assembly of claim 5 wherein said surface is a strike pad.
7. The needle assembly of claim 4 wherein said application of force is a rotational force.
8. The needle assembly of claim 4 wherein said housing mechanism further comprises a release mechanism and said application of force is the activation of said release mechanism.
9. The needle assembly of claim 4 wherein said application of force is a force applied to a syringe.
10. The needle assembly of claim 5 wherein said force applied to a syringe is a force applied to a plunger of said syringe.
11. The needle assembly of claim 4 wherein said housing comprises a first part and a second part, said second part being movable with respect to said first part, said needle being connected to said movable second part of said housing.
12. The needle assembly of claim 11 wherein said first part defines a cavity, and wherein on application of said force, said needle point retracts into said cavity.
13. The needle assembly of claim 12 wherein said first part defines a conduit, and wherein on application of said force, said needle point retracts into said conduit.
14. The needle assembly of claim 4 wherein said housing comprises:
   a first hub having a first end, a second end, and a conduit connecting the two ends, said first end defining a cavity, said first hub having an inner portion and an outer portion, said inner portion being moveable with respect to the outer portion on the application of a force;
   a second hub having a first end, a second end, and a conduit connecting the two for receiving said first hub; and
   wherein on application of a force, said inner portion is allowed to move with respect to the outer portion of the first hub causing said spring to retract the needle into said cavity defined by said first hub of said assembly.
15. The needle assembly of claim 14 wherein said spring is disposed between said first hub and said second hub.
16. The needle assembly of claim 14 wherein said needle has a first end, a second end, and a lumen running therethrough, said first end of said needle being so connected to said second end of the first hub as to provide fluid communication between the lumen and said conduit of said first hub.
17. The needle assembly of claim 14 wherein said second hub further comprises a receptacle and said first hub comprises a connector for receiving said connector when said first and second hubs are connected.
18. The needle assembly of claim 14 wherein said second hub further comprises a protruding member and said first hub further comprises a receptacle for receiving said protruding member when said first and second hubs are connected.
19. The needle assembly of claim 14 wherein said first hub further comprises a needle holder for receiving said needle.
20. The needle assembly of claim 19 wherein said first hub further comprises a depression for receiving an adhesive to secure said needle to said first hub.
21. The needle assemble of claim 14 wherein said needle assembly is provided with a connector flange for attachment of said needle assembly to a syringe.
22. The needle assembly of claim 14 wherein said first hub defines a stepped portion in said cavity for housing said spring when said first hub and said second hub are connected together.
23. The needle assembly of claim 14 further comprising a collar which seats against said second hub and said second end of said first hub.
24. The needle assembly of claim 14 wherein said application of force is a striking of the head of said needle against a surface.
25. The needle assembly of claim 14 wherein said surface is a strike pad.
26. The needle assembly of claim 14 wherein said application of force is a rotational force.
27. The needle assembly of claim 14 wherein said first hub further comprises a threaded collar and said second hub further comprises threads along an inside wall of said second hub, said threaded collar threadingly engaging said threads, and wherein said application of force is a rotational force, said rotational force causing said first part to move with respect to said second part allowing said spring to so bias the housing as to shield the needle point by said cavity defined by said first part of said housing.
28. The needle assembly of claim 14 wherein said needle assembly further comprises a release mechanism and said application of force is the activation of said release mechanism.
29. The needle assembly of claim 14 wherein said application of force is a force applied to a syringe.
30. The needle assembly of claim 29 wherein said force applied to a syringe is a force applied to a plunger of said syringe.
31. The needle assembly of claim 4 wherein said housing comprises a first hub and a second hub, said first hub having a first end and a second end; said second hub and said first end of said first hub defining a first part and said second end of said first hub defining a second part, said first part defining a cavity therein and said second part being movable with respect to said first part;
   wherein said needle is attached to said second part;
   wherein said spring is located between said first part and said second part;
   and wherein on application of said force, said second part moves with respect to said first part allowing said spring to retract the needle into said cavity defined by said first part of said housing; and
   wherein, after said retraction, said needle comes to a rest inside said cavity.
32. A syringe device comprising:
   a syringe;
   a needle assembly attached to said syringe, said needle assembly comprising: a housing; a needle attached to said housing, said needle having a needle point;
   wherein on application of a force, said needle point is retracted into said housing; and
   wherein after retraction, said needle point comes to rest inside said housing.
33. The device of claim 32 further comprising a spring connected to said housing for moving said needle, said spring retracting said needle point into said housing on the application of said force.
34. The syringe device of claim 32 wherein said application of force is a striking of the head of said needle against a surface.
35. The needle assembly of claim 32 wherein said surface is a strike pad.
36. The needle assembly of claim 32 wherein said application of force is a rotational force.
37. The needle assembly of claim 32 wherein said housing mechanism further comprises a release mechanism and said application of force is the activation of said release mechanism.
38. The needle assembly of claim 32 wherein said application of force is a force applied to a syringe.
39. The needle assembly of claim 38 wherein said force applied to a syringe is a force applied to a plunger of said syringe.
40. A method for preventing needle strike, said method comprising the steps of:
   attaching a needle assembly having a needle having a needle point to a syringe to form a syringe device;
   performing an operation with said syringe device;
   applying a force to said syringe device to retract said needle point into said needle assembly, said needle point coming to a rest inside said needle assembly.
41. The method of claim 40 wherein said step of applying a force is a striking of the head of said needle against a surface.
42. The method of claim 40 wherein said step of applying a force is applying a rotational force to said syringe device.
43. The method of claim 40 wherein said step of applying a force is the activation of a release mechanism provided on said syringe device.
44. The method of claim 40 wherein said application of force is a force applied to a syringe.
45. The needle assembly of claim 44 wherein said force applied to a syringe is a force applied to a plunger of said syringe.
46. A method of making a needle comprising the steps of:
   forming a needle having a needle point;
   forming a needle housing for receiving said needle;
   disposing a spring into said housing;
   placing said needle into said needle housing against said spring;
   placing a gasket into said housing against said needle.

While the invention has been described in conjunction with specific embodiments, it is evident that numerous alternatives, modifications, and variations will be apparent to those skilled in the art within the spirit and scope of the invention described above.

1. A needle assembly comprising:
a housing;
a needle attached to said housing, said needle having a needle point;
wherein on application of a force, said needle point is retracted into said housing; and
wherein after retraction, said needle point comes to rest inside said housing.

2. The needle assembly of claim 1 wherein said housing and said needle together form a retractable needle.

3. The needle assembly of claim 1 wherein said needle assembly further comprises a gasket around said needle, wherein on application of said force, said needle point retracts into said gasket, said gasket protecting said needle point and preventing said needle against further retraction.

4. The needle assembly of claim 1 further comprising a spring connected to said housing for moving said needle, wherein on application of said force, said spring retracts said needle point into said housing.

5. The needle assembly of claim 4 wherein said application of force is a striking of the head of said needle against a surface.

6. The needle assembly of claim 5 wherein said surface is a strike pad.

7. The needle assembly of claim 4 wherein said application of force is a rotational force.

8. The needle assembly of claim 4 wherein said housing mechanism further comprises a release mechanism and said application of force is the activation of said release mechanism.

9. The needle assembly of claim 4 wherein said application of force is a force applied to a syringe.

10. The needle assembly of claim 5 wherein said force applied to a syringe is a force applied to a plunger of said syringe.

11. The needle assembly of claim 4 wherein said housing comprises a first part and a second part, said second part being movable with respect to said first part, said needle being connected to said movable second part of said housing.

12. The needle assembly of claim 11 wherein said first part defines a cavity, and wherein on application of said force, said needle point retracts into said cavity.

13. The needle assembly of claim 12 wherein said first part defines a conduit, and wherein on application of said force, said needle point retracts into said conduit.

14. The needle assembly of claim 4 wherein said housing comprises:
a first hub having a first end, a second end, and a conduit connecting the two ends, said first end defining a cavity, said first hub having an inner portion and an outer portion, said inner portion being moveable with respect to the outer portion on the application of a force;
a second hub having a first end, a second end, and a conduit connecting the two for receiving said first hub; and
wherein on application of a force, said inner portion is allowed to move with respect to the outer portion of the first hub causing said spring to retract the needle into said cavity defined by said first hub of said assembly.

15. The needle assembly of claim 14 wherein said spring is disposed between said first hub and said second hub.

16. The needle assembly of claim 14 wherein said needle has a first end, a second end, and a lumen running therethrough, said first end of said needle being so connected to said second end of the first hub as to provide fluid communication between the lumen and said conduit of said first hub.

17. The needle assembly of claim 14 wherein said second hub further comprises a receptacle and said first hub comprises a connector for receiving said connector when said first and second hubs are connected.

18. The needle assembly of claim 14 wherein said second hub further comprises a protruding member and said first hub further comprises a receptacle for receiving said protruding member when said first and second hubs are connected.

19. The needle assembly of claim 14 wherein said first hub further comprises a needle holder for receiving said needle.

20. The needle assembly of claim 19 wherein said first hub further comprises a depression for receiving an adhesive to secure said needle to said first hub.

21. The needle assemble of claim 14 wherein said needle assembly is provided with a connector flange for attachment of said needle assembly to a syringe.

22. The needle assembly of claim 14 wherein said first hub defines a stepped portion in said cavity for housing said spring when said first hub and said second hub are connected together.

23. The needle assembly of claim 14 further comprising a collar which seats against said second hub and said second end of said first hub.

24. The needle assembly of claim 14 wherein said application of force is a striking of the head of said needle against a surface.

25. The needle assembly of claim 14 wherein said surface is a strike pad.

26. The needle assembly of claim 14 wherein said application of force is a rotational force.

27. The needle assembly of claim 14 wherein said first hub further comprises a threaded collar and said second hub further comprises threads along an inside wall of said second hub, said threaded collar threadingly engaging said threads, and wherein said application of force is a rotational force, said rotational force causing said first part to move with respect to said second part allowing said spring to so bias the housing as to shield the needle point by said cavity defined by said first part of said housing.

28. The needle assembly of claim 14 wherein said needle assembly further comprises a release mechanism and said application of force is the activation of said release mechanism.

29. The needle assembly of claim 14 wherein said application of force is a force applied to a syringe.

30. The needle assembly of claim 29 wherein said force applied to a syringe is a force applied to a plunger of said syringe.

31. The needle assembly of claim 4 wherein said housing comprises a first hub and a second hub, said first hub having a first end and a second end; said second hub and said first end of said first hub defining a first part and said second end of said first hub defining a second part, said first part defining a cavity therein and said second part being movable with respect to said first part;
wherein said needle is attached to said second part;
wherein said spring is located between said first part and said second part;
and wherein on application of said force, said second part moves with respect to said first part allowing said spring to retract the needle into said cavity defined by said first part of said housing; and
wherein, after said retraction, said needle comes to a rest inside said cavity.

32. A syringe device comprising:
a syringe;
a needle assembly attached to said syringe, said needle assembly comprising: a housing; a needle attached to said housing, said needle having a needle point;
wherein on application of a force, said needle point is retracted into said housing; and
wherein after retraction, said needle point comes to rest inside said housing.

33. The device of claim 32 further comprising a spring connected to said housing for moving said needle, said spring retracting said needle point into said housing on the application of said force.

34. The syringe device of claim 32 wherein said application of force is a striking of the head of said needle against a surface.

35. The needle assembly of claim 32 wherein said surface is a strike pad.

36. The needle assembly of claim 32 wherein said application of force is a rotational force.

37. The needle assembly of claim 32 wherein said housing mechanism further comprises a release mechanism and said application of force is the activation of said release mechanism.

38. The needle assembly of claim 32 wherein said application of force is a force applied to a syringe.

39. The needle assembly of claim 38 wherein said force applied to a syringe is a force applied to a plunger of said syringe.

40. A method for preventing needle strike, said method comprising the steps of:
attaching a needle assembly having a needle having a needle point to a syringe to form a syringe device;
performing an operation with said syringe device;
applying a force to said syringe device to retract said needle point into said needle assembly, said needle point coming to a rest inside said needle assembly.

41. The method of claim 40 wherein said step of applying a force is a striking of the head of said needle against a surface.

42. The method of claim 40 wherein said step of applying a force is applying a rotational force to said syringe device.

43. The method of claim 40 wherein said step of applying a force is the activation of a release mechanism provided on said syringe device.

44. The method of claim 40 wherein said application of force is a force applied to a syringe.

45. The needle assembly of claim 44 wherein said force applied to a syringe is a force applied to a plunger of said syringe.

46. A needle having a first end, a second end, and a lumen extending therethrough, said second end of said needle comprising a needle point and said first end of said needle comprising a stop.

47. The needle of claim 46 further comprising a housing, said needle retracting into said housing on the application of a force, said needle point coming to a rest inside said housing.

48. The needle of claim 47 further comprising a means for holding said needle in an extended position relative to said housing.

49. The needle of claim 47 further comprising a means for retracting said needle into said housing on the application of a force, said needle point coming to a rest inside said housing.
